(19) United States
(12) Patent Application Publication
(10) Pub. No.: US 2019/0256459 A1
(43) Pub. Date: Aug. 22, 2019

(54) NOVEL PROCESS FOR THE PREPARATION OF BELINOSTAT

(71) Applicant: Biophore India Pharmaceuticals PVT. Ltd., Hyderabad (IN)

(72) Inventors: Manik Reddy PULLAGURLA, Hyderabad (IN); Mecheril Valsan NANDA KUMAR, Hyderabad (IN); Bhaskar Reddy PITTA, Hyderabad (IN); Jagadeesh Babu RANGISETTY, Hyderabad (IN)

(21) Appl. No.: 16/302,103

(22) PCT Filed: May 1, 2017
(86) PCT No.: PCT/IN2017/050154

§ 371 Date: Nov. 16, 2018

(30) Foreign Application Priority Data
May 17, 2016 (IN) 201641017008

(51) Int. Cl. C07C 303/38; C07C 303/40; C07C 303/44; C07C 201/12; C07C 209/32

(52) U.S. Cl. CPC C07C 303/38; C07C 303/40; C07C 205/56; C07C 201/12; C07C 209/325; C07C 303/44

(57) ABSTRACT

The present invention provides a novel and commercially viable process with high yield for the preparation of (E)-N-hydroxy-3-(3-phenylsulfamoyl-phenyl)-acrylamide, also known as Belinostat (I). The invention also provides process for purification and novel crystalline form of Belinostat in substantially pure form.

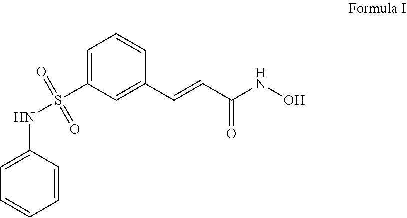 [SMILES: O=C(/C=C/c1cccc(S(=O)(=O)Nc2ccccc2)c1)NO]

### CROSS REFERENCE

This PCT application claims priority from Indian Patent Application No. 201641017008 filed on May 17, 2016.

### FIELD OF THE INVENTION

The invention relates to a novel process for the preparation of Belinostat. More particularly, the invention relates a novel and commercially viable synthetic process for the preparation of (E)-N-hydroxy-3-(3-phenylsulfamoyl-phenyl)-acrylamide, also known as Belinostat structurally represented by the below formula I. The invention also relates to methods of purification of Belinostat, a novel crystalline form of it and process for preparation thereof. The purification process provides substantially pure Belinostat having purity of greater than 98% and impurities less than 0.05% (w/w) by HPLC.

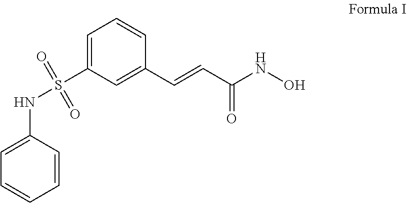 [SMILES: O=C(/C=C/c1cccc(S(=O)(=O)Nc2ccccc2)c1)NO]

### BACKGROUND OF THE INVENTION

The compound (E)-N-hydroxy-3-(3-phenylsulfamoyl-phenyl)-acrylamide, also known as Belinostat is represented by the formula I:

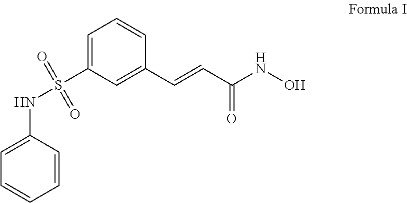 [SMILES: O=C(/C=C/c1cccc(S(=O)(=O)Nc2ccccc2)c1)NO]

Belinostat is indicated for the treatment of patients with relapsed or refractory peripheral T-cell lymphoma (PTCL).The compound of formula I was first described in U.S. Pat. No. 7,183,298 which discloses a multi-step method of synthesis, schematically as represented in prior art process Scheme-1 below.

PRIOR ART PROCESS SCHEME-1

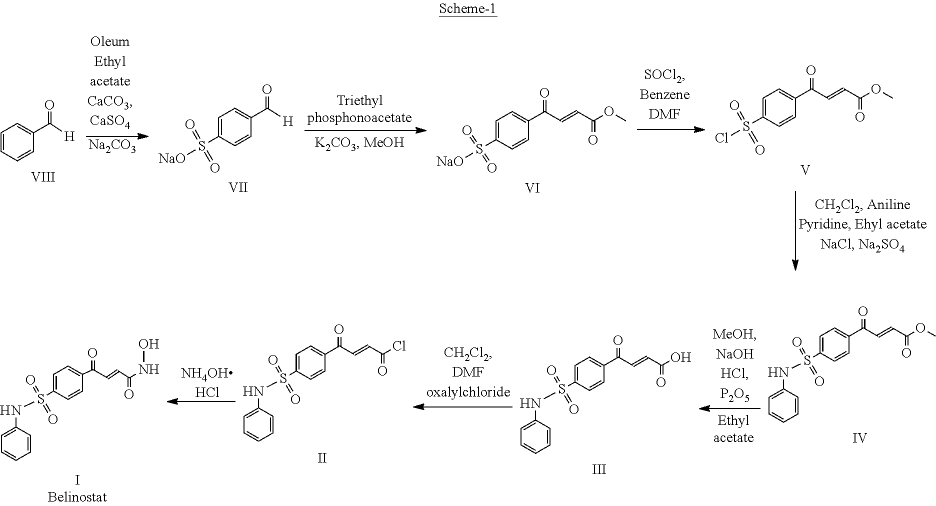

Another process has been disclosed in the U.S. Pat. No. 8,642,809 for the preparation of Belinostat as shown in prior art process Scheme-2 below.

PRIOR ART PROCESS SCHEME-2

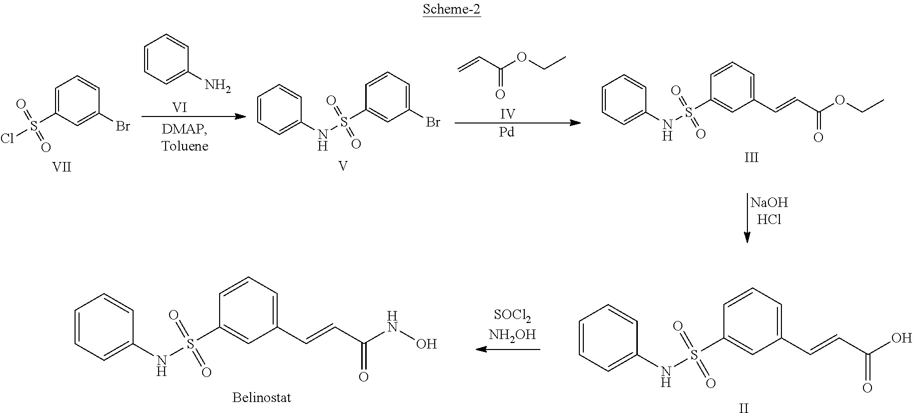

The Belinostat preparation method reported in U.S. Pat. No. 7,183,298 (Scheme-1 above), suffers from one or more disadvantages or limitations viz., the process involves sulfonylation of the benzaldehyde with extremely corrosive oleum, which makes the process difficult for industrial level scale-up. In addition, large excess of base (CaCO₃) is required for the neutralization of oleum, as a result large amount of inorganic salts are formed. Separation of these inorganic salts from the obtained 3-benzenesulfonic acid sodium salt is very difficult because of its high-water solubility, and this leads to inconsistent assay of the compound. In this process overall yield of Belinostat is about 12%. The second method reported in U.S. Pat. No. 8,642,809 (Scheme-2 above), requires Palladium catalyst which makes the process expensive. The other alternative method reported by Lei Yang et. al., in Synthetic Communications 2010, 40, 2520-2524, involves extensive column purification of Methyl (2E)-3-{3-[(Phenylamino)sulfonyl]phenyl}prop-2-enoate, which is a key intermediate for the preparation of Belinostat.

The present invention overcomes the major problems encountered in the prior arts by avoiding hazardous chemicals like oleum and tedious column purifications. The present invention provides a safe, cost-effective, time saving and reliable process for the preparation of Belinostat in bulk scale and in substantially pure form.

### OBJECT OF THE INVENTION

One object of the invention is to provide a novel process for the preparation of Belinostat of formula I.

Another object of the invention is to provide an improved process for the preparation of Belinostat of formula I with high overall yield and purity.

Another object of the invention is to provide methods of purification of Belinostat.

Another object of the invention is to provide substantially pure Belinostat compound of formula I with purity greater than 98% and impurities less than 0.05% (w/w) by HPLC.

Another object of the invention is to provide a novel crystalline form of Belinostat of formula I and process for preparation thereof.

Yet, another object of the invention is to provide a cost effective, commercially viable synthetic process for the preparation of Belinostat.

### SUMMARY OF THE INVENTION

Accordingly, a novel process for the preparation of Belinostat of formula I as depicted in Scheme-3 and Scheme-3A is provided.

In one aspect of the invention, synthesis of Belinostat is reported which results in significantly high overall yield, greater than 40%. The overall process for the preparation of Belinostat involves the steps of:
(a) reacting 3-nitrobenzaldehyde of formula VIII with suitable Wittig reagent to provide (E)-ethyl 3-(3-nitrophenyl)acrylate of formula VI;

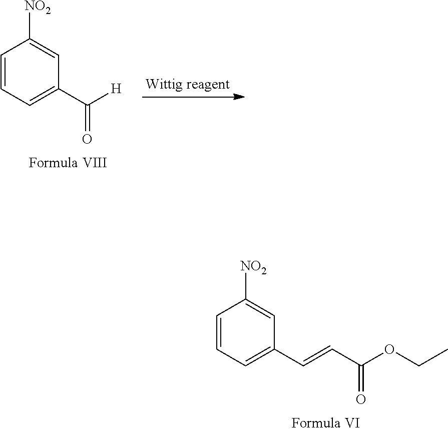 [SMILES: O=Cc1cccc([N+](=O)[O-])c1 ; CCOC(=O)/C=C/c1cccc([N+](=O)[O-])c1]

(b) reducing (E)-ethyl 3-(3-nitrophenyl)acrylate of formula VI with suitable reducing agent to provide (E)-ethyl 3-(3-aminophenyl)acrylate of formula V;

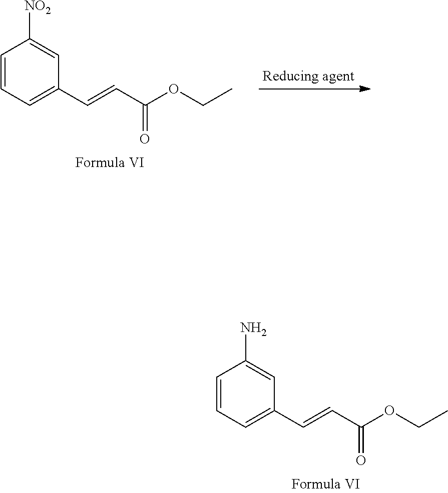 [SMILES: CCOC(=O)/C=C/c1cccc([N+](=O)[O-])c1 ; CCOC(=O)/C=C/c1cccc(N)c1]

(c) diazotizing (E)-ethyl 3-(3-aminophenyl)acrylate of formula V followed by chlorosulphonating with suitable chlorosulphonating reagent to provide (E)-ethyl 3-(3-(chlorosulfonyl)phenyl)acrylate of formula IV;

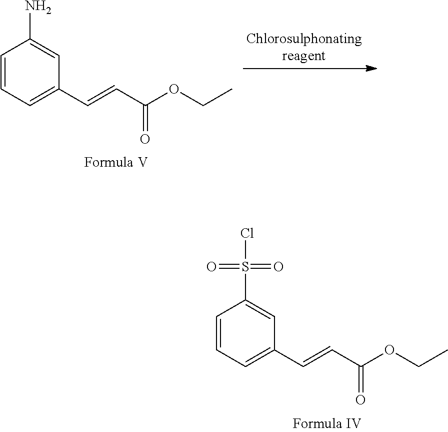 [SMILES: CCOC(=O)/C=C/c1cccc(N)c1 ; CCOC(=O)/C=C/c1cccc(S(=O)(=O)Cl)c1]

(d) coupling (E)-ethyl 3-(3-(chlorosulfonyl)phenyl)acrylate of formula IV with aniline to provide (E)-3-(3-(N-phenylsulfamoyl)phenyl)acryli c acid of formula III;

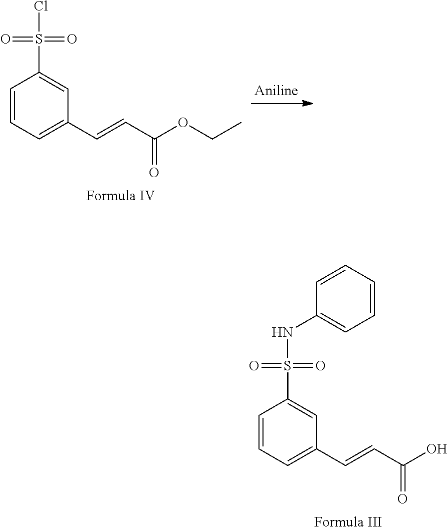 [SMILES: CCOC(=O)/C=C/c1cccc(S(=O)(=O)Cl)c1 ; O=C(O)/C=C/c1cccc(S(=O)(=O)Nc2ccccc2)c1]

(e) converting (E)-3-(3-(N-phenylsulfamoyl)phenyl)acrylic acid of formula III to acid chloride with suitable chlorinating agent to provide (E)-3-(3-(N-phenylsulfamoyl) phenyl) acryloyl chloride of formula II;

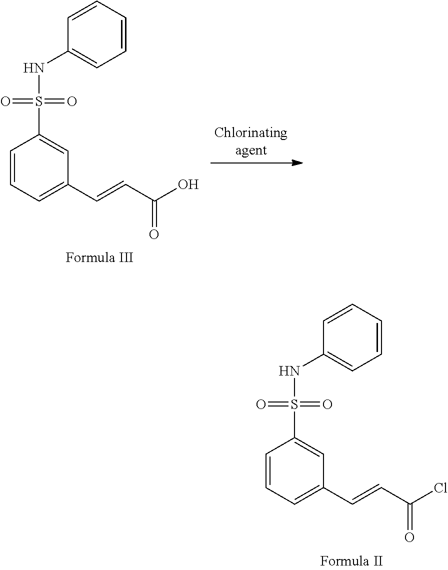 [SMILES: O=C(O)/C=C/c1cccc(S(=O)(=O)Nc2ccccc2)c1 ; O=C(Cl)/C=C/c1cccc(S(=O)(=O)Nc2ccccc2)c1]

(f) amino hydroxylating (E)-3-(3-(N-phenylsulfamoyl) phenyl) acryloyl chloride of formula II with hydroxyl amine hydrochloride to provide crude Belinostat of formula I; and

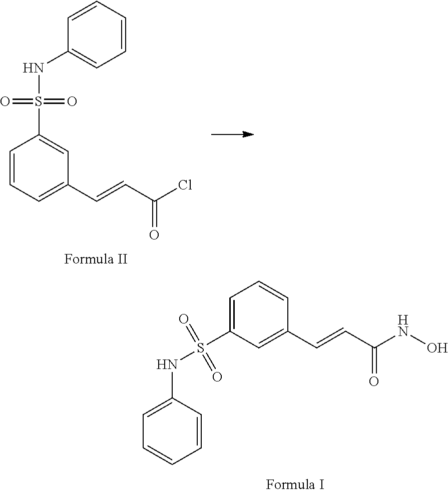 [SMILES: O=C(Cl)/C=C/c1cccc(S(=O)(=O)Nc2ccccc2)c1 ; O=C(/C=C/c1cccc(S(=O)(=O)Nc2ccccc2)c1)NO]

(g) optionally, purifying the compound of formula I by re-crystallizing from suitable solvents to provide substantially pure Belinostat of formula I.

The suitable Wittig reagent in step (a) is selected from the group comprising of Ethyl (triphenylphosphoranylidene) acetate, tert-Butoxycarbonylmethylene) triphenylphosphorane, benzyl (triphenylphosphoranylidene) phenylphosphoranylidene) acetate, methyl (triphenylphosphoranylidene) acetate, Triethylphosphonoacetate and trimethylphosphonoacetate. In one preferred embodiment, the Wittig reagent used in step (a) is triethylphosphonoacetate of formula VII.

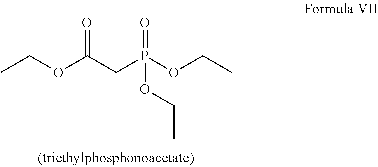

The process step (a) is carried out in the presence of suitable base selected from suitable inorganic base selected from the group comprising caesium carbonate (Cs₂CO₃), potassium carbonate (K₂CO₃), sodium carbonate (Na₂CO₃),silver carbonate (Ag₂CO₃),tripotassium phosphate (K₃PO₄), sodium hydroxide (NaOH), potassium hydroxide (KOH), caesium hydroxide (CsOH), potassium methoxide (KOMe), sodium methoxide (NaOMe),sodium ethoxide (NaOEt), lithium tert-butoxide (LiOtBu), sodium tert-butoxide (NaOtBu),potassium tert-butoxide (KOtBu), Sodium bis(trimethylsilyl)amide (NaHMDS), potassium bis(trimethylsilyl)amide (KHMDS), sodium hydride (NaH); and suitable organic base selected from the group comprising of pyridine, triethyl amine, leutidine, (1,4-diazabicyclo[2.2.2]octane) (DABCO), 1,8-Diazabicyclo[5.4.0]undec-7-ene (DBU), N,N-diisopropylethylamine (DIPEA), butyl lithium (BuLi), lithium diisopropylamide (LDA), 1,2,2,6,6-pentamethylpipiridine, 1,1,3,3-tetramethylguanidine, diispropyl ethyl amine (iPr₂Net) and tributyl amine (NBu₃). In one preferred embodiment the suitable base used in step (a) is potassium carbonate (K₂CO₃).

The suitable reducing agent used in step (b) is Tin chloride-dihydrate.

The suitable chlorosulphonating reagent used in step (c) is selected from copper (I) chloride or copper (II) chloride. In one preferred embodiment copper (I) chloride is used.

The suitable chlorosulphonating reagent in step (c) is used in combination with sulphur dioxide gas. Further the step (c) is carried out in the presence of a suitable acid, preferably hydrochloric acid (HCl). The reaction temperature in step (c) ranges from −10 to 0° C., preferably −5 to 0° C.

The step (d) is carried out in the presence of suitable bases selected from the group comprising triethyl amine, pyridine, metal carbonates, metal hydroxides, metal alkoxides and dimethyl amino pyridine. In one preferred embodiment, the base used in step (d) is dimethyl amino pyridine (DMP).

The suitable acid chlorinating agent used in step (e) is selected from the group comprising thionyl chloride and oxalylchloride. In one preferred embodiment the acid chlorinating agent used in step (e) is oxalylchloride. In another embodiment step (e) is carried out in presence of catalytic amount of dimethylformamide (DMF).

One or more of the process steps (a), (b), (c), (d), (e) and (f) can be carried out in presence of suitable solvent selected from the group comprising water, alcohols, hydrocarbons, aromatic hydrocarbons, chloro hydrocarbons, esters, ketones, ethers, nitriles, acetic acid or mixtures thereof.

In one embodiment, the step (a) is carried out in presence of solvent water (DM water).

In one embodiment, the step (b) is carried out in presence of suitable solvent selected from alcohols comprising ethanol, methanol, propanol, isopropanol and mixtures thereof. Preferably ethanol (EtOH) is used.

In one embodiment, the step (c) is carried out in presence of solvent aqueous acetic acid (Aq. AcOH).

In one embodiment, the step (d) is carried out in presence of solvent dichloromethane (DCM).

In one embodiment, the step (e) is carried out in presence of solvent dichloromethane (DCM).

In one embodiment, the step (f) is carried out in presence of solvent tetrahydrofuran (THF).

The suitable solvent in step (g) is selected from the group comprising alkanes, alcohols, water, acetonitrile, tetrahydrofuran, acetone, ethyl acetate, dichloromethane, methyl tertiary butyl ether, diethyl ether, isopropyl ether, acetic acid or mixtures thereof.

Thus in one preferred embodiment, the process provides crude Belinostat of formula I comprising the steps of:
(a) reacting 3-nitrobenzaldehyde of formula VIII with triethylphosphonoacetate in the presence of Potassium carbonate and water to provide (E)-ethyl 3-(3-nitrophenyl)acrylate of formula VI;
(b) reducing (E)-ethyl 3-(3-nitrophenyl)acrylate of formula VI with tin chloride-dihydrate in presence of ethanol solvent to provide compound of formula V;
(c) chlorosulfonating (E)-ethyl 3-(3-aminophenyl)acrylate of formula V with copper (I) chloride by passing sulphur dioxide gas in presence of mixture of acetic acid and hydrochloric acid to provide formula IV;
(d) coupling (E)-ethyl 3-(3-(chlorosulfonyl)phenyl)acrylate of formula IV with aniline in the presence of 4-dimethylaminopyridine to provide formula III;
(e) chlorinating (E)-3-(3-(N-phenylsulfamoyl)phenyl)acrylic acid of formula III with oxalylchloride in the presence of dimethylformamide to give formula II;
(f) amino hydroxylating (E)-3-(3-(N-phenylsulfamoyl) phenyl) acryloyl chloride of formula II with hydroxyl amine hydrochloride to provide crude Belinostat of formula I.

The above process optionally, further comprises a purification step (g).

In yet another object of the invention, methods of purification of Belinostat to obtain a substantially pure compound, whose purity is greater than or equal to 99% is reported.

Thus the invention provides a process for the purification of Belinostat of formula I, comprising the steps of:
(a) suspending crude Belinostat of formula I in suitable solvents selected from ethyl acetate, methanol, ethanol, isopropanol, n-butanol, water, methyl tertiary butyl ether, acetone, diethyl ether, isopropyl ether, dichloromethane, acetonitrile, acetic acid and tetrahydrofuran, toluene, cyclohexane solvents or mixtures thereof;
(b) heating the reaction mixture to a temperature ranging from 25°-65° C. for 1 to 5 hrs;
(c) cooling the reaction mixture to 0-5° C.;
(d) filtering the solid separated under vacuum; and
(e) optionally, washing the solid with the mixture of suitable solvents selected from ethyl acetate, methanol, ethanol, isopropanol, n-butanol, water, methyl tertiary butyl ether, acetone, diethyl ether, isopropyl ether, dichloromethane, acetonitrile, acetic acid and tetrahydrofuran, toluene, cyclohexane solvents or mixtures thereof at 0-5° C.

In one embodiment, the suitable solvent used in step (a) of above said purification process is a mixture of solvents selected from combinations: ethyl acetate and methanol (4:1) mixture, ethanol and ethyl acetate mixture (2:4) mixture, isopropyl alcohol and ethyl acetate (3:4) mixture, dichloromethane and ethyl acetate (0.5:6) mixture and Isopropyl alcohol and Tetrahydrofuran (1:3) mixture.

The pure Belinostat of formula I obtained by above process after purification is in crystalline form designated as form 1, characterized by an X-ray diffraction pattern comprising one or more of the reflections at: 4.5, 13.5, 14.6, 18.0, 20.7, 21.1 and 26.3±0.2 degrees 2 theta; which is further characterized by an X-ray powder diffraction pattern comprising one or more of the reflections at: 8.9, 11.1, 15.4, 16.5, 22.4, 22.6 & 28.0±0.2 degrees 2 theta.

In one aspect of the invention, a crystalline form 1 of Belinostat is reported whose X-ray diffraction pattern is represented by FIG. 1 and XRPD values are tabulated in Table-1. Belinostat obtained by the process of the present invention is analysed by IR, DSC and XRD.

A crystalline Belinostat of formula I designated as crystalline form 1, characterized by an X-ray diffraction pattern comprising one or more of the reflections at: 4.5, 13.5, 14.6, 18.0, 20.7, 21.1 and 26.3±0.2 degrees 2 theta; which is further characterized by an X-ray powder diffraction pattern comprising one or more of the reflections at: 8.9, 11.1, 15.4, 16.5, 22.4, 22.6 & 28.0±0.2 degrees 2 theta.

Crystalline Belinostat of formula I designated as crystalline form 1, characterized by XRPD FIG. 1 and XRPD value as depicted in table-1, DSC thermogram as in FIG. 2 and IR spectrum as in FIG. 3.

The crystalline Belinostat obtained after above purification process is substantially pure having purity of greater than 98%, preferably equal to or greater than 99% and impurities less than 0.05% (w/w), preferably less than 0.03% (w/w) by HPLC.

Substantially pure Belinostat compound of formula I having purity of greater than 98%, preferably equal to or greater than 99% and impurities less than 0.05% (w/w), preferably less than 0.03% (w/w) by HPLC.

### BRIEF DESCRIPTION OF THE DRAWINGS

FIG. 1: Illustrates the X-Ray powder diffraction (XRPD) pattern of crystalline form 1 of Belinostat

FIG. 2: Illustrates the Differential scanning calorimetry (DSC) graph of crystalline form 1 of Belinostat

FIG. 3: Illustrates the IR spectrum of crystalline form 1 of Belinostat

### DETAILED DESCRIPTION OF THE INVENTION

The present invention relates to a novel process for the preparation of Belinostat. More particularly, the invention relates a novel and commercially viable synthetic process for the preparation of (E)-N-hydroxy-3-(3-phenylsulfamoyl-phenyl)-acrylamide, also known as Belinostat structurally represented by the below formula I.

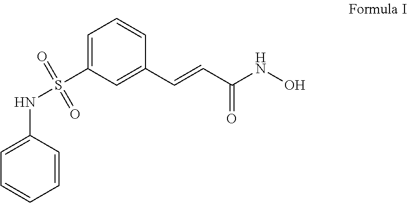 [SMILES: O=C(/C=C/c1cccc(S(=O)(=O)Nc2ccccc2)c1)NO]

The invention also relates to methods of purification of Belinostat, a novel crystalline form of it and process for preparation thereof. The invention provides Belinostat of formula I in significantly high yield, substantially pure form, having purity of greater than 98%, preferably greater than 99% and impurities less than 0.05% (w/w) by HPLC, preferably less than 0.03% (w/w) by HPLC.

The general synthetic scheme of the present invention for the preparation of Belinostat of formula I is schematically presented in below general Scheme 3.

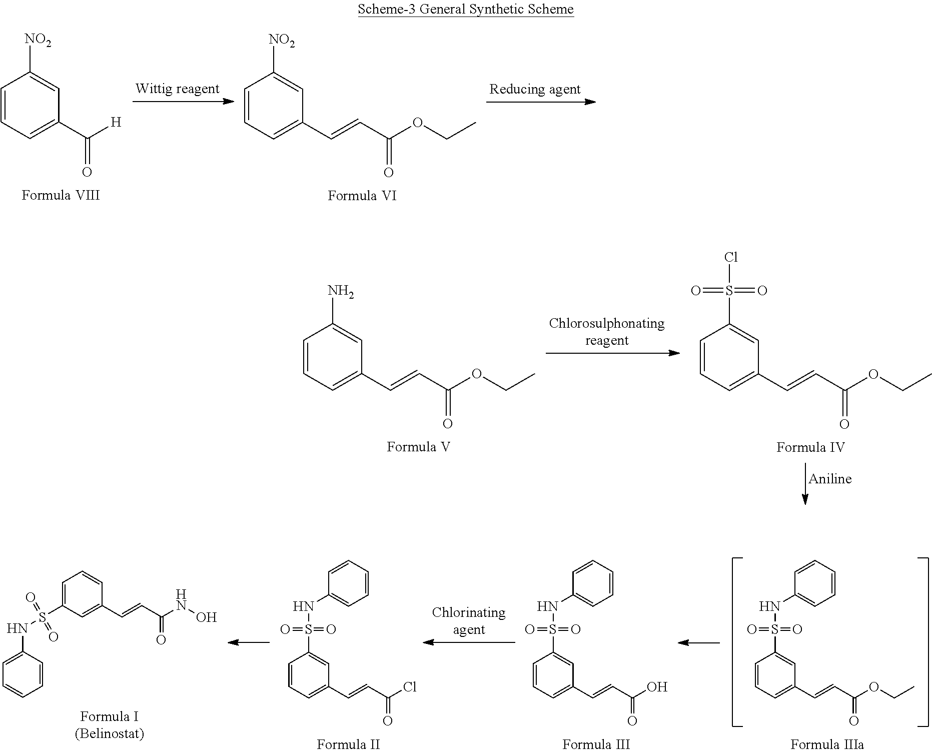

In one aspect, the invention provides a process for synthesis of Belinostat which results in significantly high overall yield, greater than 40%. The overall process for the preparation of Belinostat is as presented in Scheme-3 above, which involves following process steps (a) to (f):
(a) reacting 3-nitrobenzaldehyde of formula VIII with suitable Wittig reagent to provide (E)-ethyl 3-(3-nitrophenyl)acrylate of formula VI;
(b) reducing (E)-ethyl 3-(3-nitrophenyl)acrylate of formula VI with suitable reducing agent to provide (E)-ethyl 3-(3-aminophenyl)acrylate of formula V;
(c) diazotizing (E)-ethyl 3-(3-aminophenyl)acrylate of formula V followed by chlorosulphonating with suitable chlorosulphonating reagent to provide (E)-ethyl 3-(3-(chlorosulfonyl)phenyl)acrylate of formula IV;
(d) coupling (E)-ethyl 3-(3-(chlorosulfonyl)phenyl)acrylate of formula IV with aniline to provide (E)-3-(3-(N-phenylsulfamoyl)phenyl)acrylic acid of formula III;
(e) converting (E)-3-(3-(N-phenylsulfamoyl)phenyl)acrylic acid of formula III to acid chloride with suitable chlorinating agent to provide (E)-3-(3-(N-phenylsulfamoyl) phenyl) acryloyl chloride of formula II; and
(f) amino hydroxylating (E)-3-(3-(N-phenylsulfamoyl) phenyl) acryloyl chloride of formula II with hydroxyl amine hydrochloride to provide crude Belinostat of formula I.

Optionally, the above produced crude Belinostat of formula I may further comprise a purification step (g) to provide substantially pure Belinostat of formula I.
(g) optionally, purifying the compound of formula I by re-crystallizing from suitable solvents to provide substantially pure Belinostat of formula I.

The details reaction conditions of above general reaction steps are as illustrated below:

Step-(a): PREPARATION OF (E)-ETHYL 3-(3-NITROPHENYL) ACRYLATE (Formula VI)

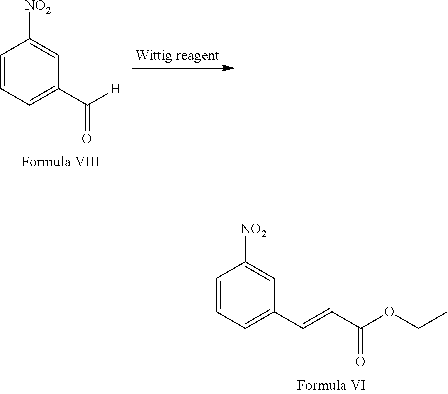 [SMILES: O=Cc1cccc([N+](=O)[O-])c1 ; CCOC(=O)/C=C/c1cccc([N+](=O)[O-])c1]

In this step 3-nitrobenzaldehyde (VIII) is charged with a base and optionally in presence of a solvent at 25-30° C. Then slowly the Witting reagent is added for a period of 20-30 min and maintained for 1-4 hrs. After completion of reaction, the solid is filtered under vacuum, preferably at 25-30° C. and is washed with hydrochloric acid followed by washing with solvent and obtained solid is dried to get compound of formula VI.

The suitable Wittig reagent in step (a) is selected from the group comprising of Ethyl (triphenylphosphoranylidene) acetate, tert-Butoxycarbonylmethylene) triphenylphosphorane, benzyl (triphenylphosphoranylidene) acetate, methyl (triphenylphosphoranylidene) acetate, Triethylphosphonoacetate and trimethylphosphonoacetate. In one preferred embodiment, the Wittig reagent used in step (a) is triethylphosphonoacetate of formula VII.

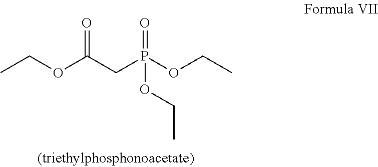

The suitable base can be selected from inorganic base and organic base. Suitable inorganic base is selected from the group comprising caesium carbonate (Cs₂CO₃), potassium carbonate (K₂CO₃), sodium carbonate (Na₂CO₃), silver carbonate (Ag₂CO₃),tripotassium phosphate (K₃PO₄), sodium hydroxide (NaOH), potassium hydroxide (KOH), caesium hydroxide (CsOH), potassium methoxide (KOMe), sodium methoxide (NaOMe),sodium ethoxide (NaOEt), lithium tert-butoxide (LiOtBu), sodium tert-butoxide (NaOtBu),potassium tert-butoxide (KOtBu), Sodium bis(trimethylsilyl)amide (NaHMDS), potassium bis(trimethylsilyl)amide (KHMDS), sodium hydride (NaH). Suitable organic base is selected from the group comprising of pyridine, triethyl amine, leutidine, (1,4-diazabicyclo[2.2.2]octane) (DABCO), 1,8-Diazabicyclo[5.4.0]undec-7-ene (DBU), N,N-diisopropylethylamine (DIPEA), butyl lithium (BuLi), lithium diisopropylamide (LDA), 1,2,2,6,6-pentamethylpipiridine, 1,1,3,3-tetramethylguanidine, diispropyl ethyl amine (iPr₂Net) and tributyl amine (NBu₃). In one preferred embodiment of the invention, the base selected is potassium carbonate (K₂CO₃).

In one embodiment step (a) is carried out without a solvent. In another embodiment step (a) is carried out in presence of a solvent. The suitable solvent is selected from the group comprising water, alcohols, hydrocarbons, aromatic hydrocarbons, chloro hydrocarbons, esters, ketones, ethers, nitriles, acetic acid or mixtures thereof. In one preferred embodiment, the suitable solvent used in step (a) is water.

Step-(b): PREPARATION OF (E)-ETHYL 3-(3-AMINOPHENYL) ACRYLATE (Formula V)

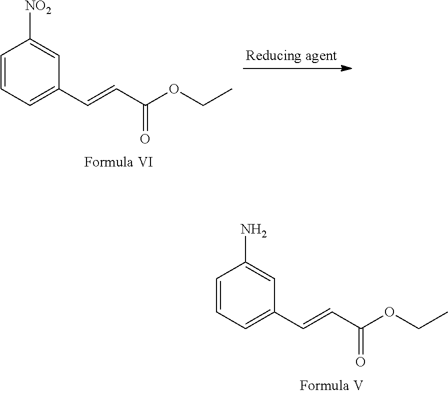 [SMILES: CCOC(=O)/C=C/c1cccc([N+](=O)[O-])c1 ; CCOC(=O)/C=C/c1cccc(N)c1]

In this step, (E)-ethyl 3-(3-nitrophenyl) acrylate (VI) is dissolved in a solvent and treated with the reducing agent at 25-30° C. Then the temperature of the reaction mass is maintained at 75-85° C., preferably 80-82° C. for 3-5 hrs followed by cooling to 25-30° C. After further pH neutralization and washings the mass is dried and organic layer is distilled under vacuum followed by further washing and drying to give compound of formula V.

The suitable reducing agent in step (b) is Tin chloride-dihydrate.

The suitable solvent in step (b) is selected from the group comprising water, alcohols, hydrocarbons, aromatic hydrocarbons, chloro hydrocarbons, esters, ketones, ethers, nitriles, acetic acid or mixtures thereof. In one preferred embodiment, step (b) is carried out in presence of suitable solvent selected from alcohols comprising ethanol, methanol, propanol, isopropanol and mixtures thereof. More preferably the ethanol is used.

Step-(c): PREPARATION OF (E)-ETHYL 3-(3-(CHLOROSULFONYL) PHENYL) ACRYLATE (Formula IV)

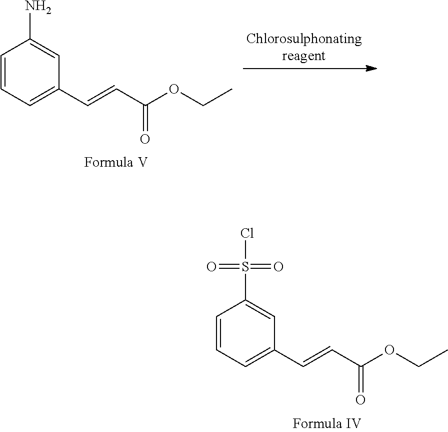 [SMILES: CCOC(=O)/C=C/c1cccc(N)c1 ; CCOC(=O)/C=C/c1cccc(S(=O)(=O)Cl)c1]

In this step, (E)-ethyl 3-(3-aminophenyl) acrylate (V) is treated with a chlorosulphonating reagent to give compound of formula IV. In step c) the reaction temperature ranges from −10 to 0° C., preferably −5 to 0°.

Compound of formula V in presence of a solvent and suitable acid such as HCl at 25-30° C. are added and cooled to −5 to 0° C., followed by addition of aqueous sodium nitrite solution at −5 to 0° C. and maintained for 30-40 min. To this a mixture of solvent and chlorosulphonating reagent solution saturated with sulphur dioxide gas is added at −5 to 0° C. The reaction mass is maintained at 5 to 0° C., followed by quenching with ice, stirring at −5 to 0° C., filtration and drying to obtain compound of formula IV.

Suitable chlorosulphonating reagent is either copper (I) chloride or copper (II) chloride. In one embodiment the chlorosulphonating reagent is used in combination with sulphur dioxide gas. In one preferable embodiment, the reagent used is copper (I) chloride in combination with sulphur dioxide gas.

The suitable solvent is selected from the group comprising water, alcohols, hydrocarbons, aromatic hydrocarbons, chloro hydrocarbons, esters, ketones, ethers, nitriles, acetic acid or mixtures thereof. In one preferred embodiment, the suitable solvent used is acetic acid.

In one preferred embodiment, the suitable acid used in step (c) is hydrochloric acid (HCl).

Step-(d): PREPARATION OF (E)-3-(3-(N-PHENYLSULFAMOYL) PHENYL) ACRYLIC ACID (Formula III)

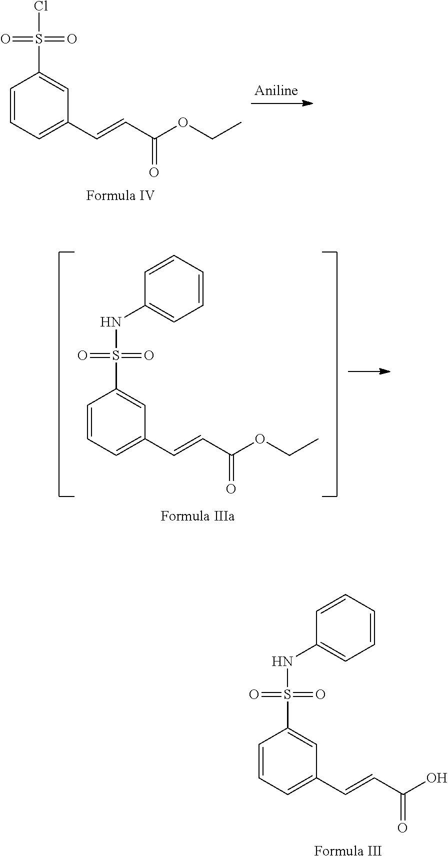

Step (d) involves coupling of Formula IV with Aniline to initially provide an intermediate compound of formula Ma which is further converted to compound of formula III.

In this step to a mixture aniline in solvent and a base, (E)-ethyl 3-(3-aminophenyl) acrylate (IV) dissolved in solvent is added. After completion of the reaction the intermediate compound of formula IIIa is obtained which on further treatment with solvent, acid, washings, treatment with charcoal, filtering and drying provides compound of formula III.

In step (d) suitable bases used for the coupling of (E)-ethyl 3-(3-(chlorosulfonyl) phenyl)acrylate of formula IV with aniline is selected from the group comprising triethyl amine, pyridine, metal carbonates, metal hydroxides, metal alkoxides and dimethyl amino pyridine (DMP). In one preferred embodiment the base used in step (d) is DMP.

Step (d) can be performed in presence of a suitable solvent selected from the group comprising water, alcohols, hydrocarbons, aromatic hydrocarbons, chloro hydrocarbons, esters, ketones, ethers, nitriles, acetic acid or mixtures thereof.

In one preferred embodiment the solvent used in step (d) is dichloromethane (DCM).

Step-(e): PREPARATION OF (E)-3-(3-(N-PHENYLSULFAMOYL) PHENYL) ACRYLOYL CHLORIDE (Formula II)

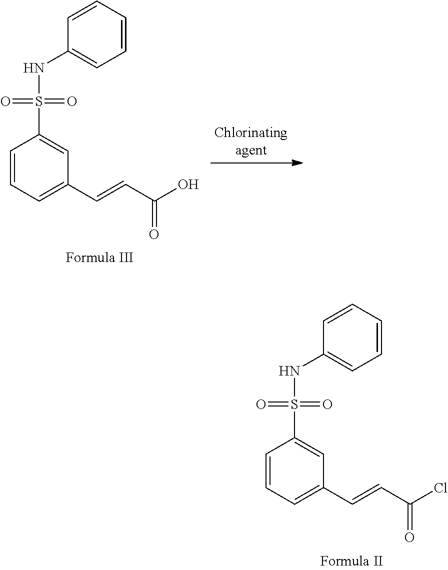 [SMILES: O=C(O)/C=C/c1cccc(S(=O)(=O)Nc2ccccc2)c1 ; O=C(Cl)/C=C/c1cccc(S(=O)(=O)Nc2ccccc2)c1]

In this step (E)-3-(3-(N-phenylsulfamoyl) phenyl) acrylic acid (III) is dissolved in suitable solvent or mixture of solvents and suitable acid chlorinating agent is slowly added at 25-30° C. followed by stirring and distilling the solvent under vacuum to obtain crude product (formula II) mass which is used in next step.

In step (e) suitable acid chlorinating agent is selected from the group comprising thionyl chloride and oxalylchloride. In one preferred embodiment chlorinating agent used is oxalylchloride.

Suitable solvent is selected from the group comprising water, alcohols, hydrocarbons, aromatic hydrocarbons, chloro hydrocarbons, esters, ketones, ethers, nitriles, acetic acid or mixtures thereof. In one preferred embodiment the solvent used in step (e) is tetrahydrofuran (THF).

In another embodiment step (e) can be carried out in presence of catalytic amount of dimethylformamide (DMF).

Step-(f): PREPARATION OF (E)-N-HYDROXY-3-(3-PHENYLSULFAMOYL-PHENYL)-ACRYLAMIDE (Formula I, Crude Belinostat)

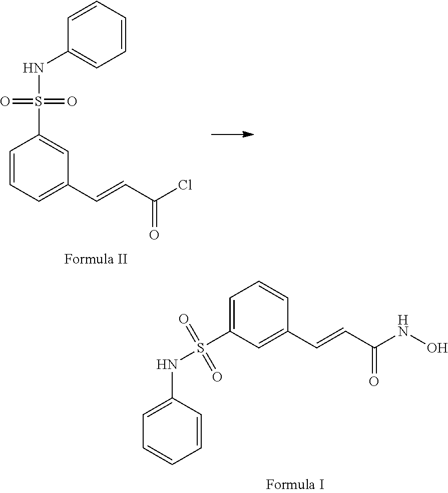 [SMILES: O=C(Cl)/C=C/c1cccc(S(=O)(=O)Nc2ccccc2)c1 ; O=C(/C=C/c1cccc(S(=O)(=O)Nc2ccccc2)c1)NO]

In this step product of step (e) is dissolved in solvent and treated with hydroxyl amine hydrochloride dissolved in solvent to provide crude Belinostat of formula I.

In one embodiment, suitable solvent used in step (f) is selected from the group comprising water, alcohols, hydrocarbons, aromatic hydrocarbons, chloro hydrocarbons, esters, ketones, ethers, nitriles, acetic acid or mixtures thereof. Preferably in one embodiment solvent used in step (f) is tetrahydrofuran (THF).

Step-(g): PURIFICATION OF (E)-N-HYDROXY-3-(3-PHENYLSULFAMOYL-PHENYL)-ACRYLAMIDE (I) OR BELINOSTAT

The purification is performed by re-crystallization by suspending crude Belinostat in a suitable solvent or mixture of solvents. In step (g) the crude Belinostat obtained in the above process is further purified from solvents selected from the group comprising alkanes like hexanes, toluene, cyclohexane; alcohols like methanol, ethanol, n-butanol, isopropyl alcohol; water; acetonitrile; tetrahydrofuran; acetone; ethyl acetate; dichloromethane , methyl tertiary butyl ether, diethyl ether, isopropyl ether, acetic acid or mixtures thereof.

Crude solid is suspended in a solvent or mixture of solvents and stirred for 12 to 18 hrs, preferably 15 hrs at 25-30° C. The reaction mixture is cooled to 0-5° C. and maintained for 3-5 hrs followed by filtration, washing and drying under vacuum to obtain the pure Belinostat of formula I. In one preferred embodiment the purification is carried out in a suitable mixture of two or more of above said solvents. In one preferred embodiment, the present invention provides a process for preparation of Beleinostat, schematically as represented in below Scheme-3A:

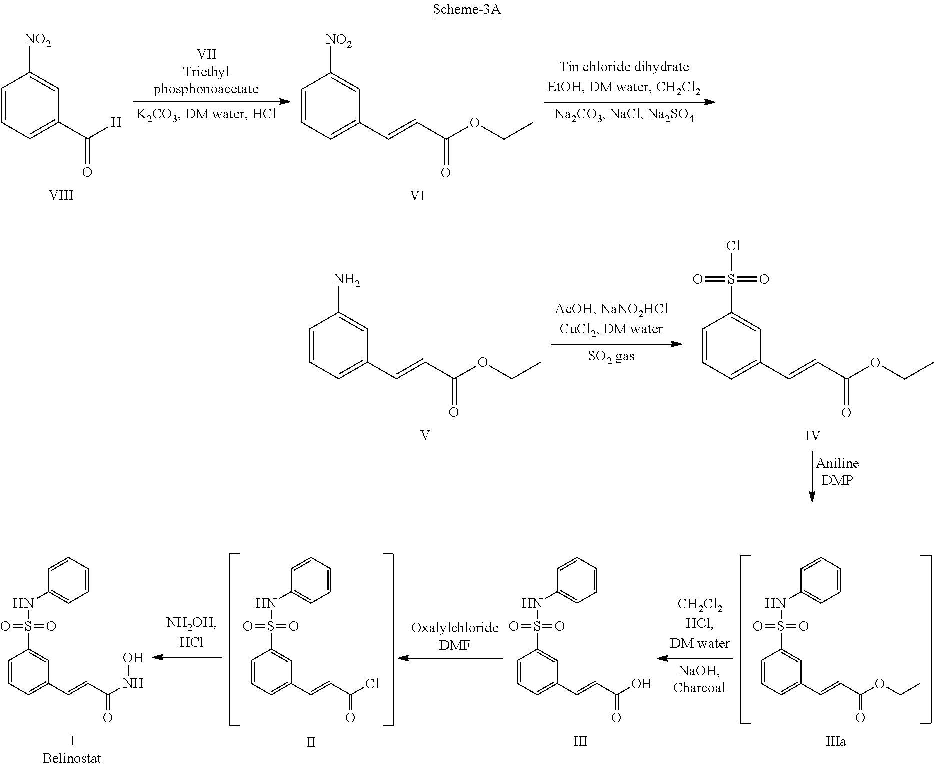

The present invention relates to an improved process for the preparation of Belinostat in significantly high yield and substantially pure form. The process of the present invention comprises:

a) reacting 3-nitrobenzaldehyde of formula VIII

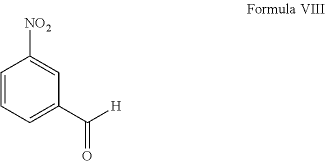 [SMILES: O=Cc1cccc([N+](=O)[O-])c1]

with suitable Wittig reagent triethylphosphonoacetate of formula VII

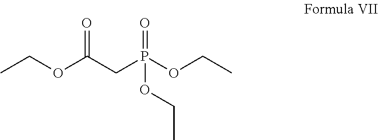

in the presence of suitable base to give (E)-ethyl 3-(3-nitrophenyl)acrylate of formula VI;

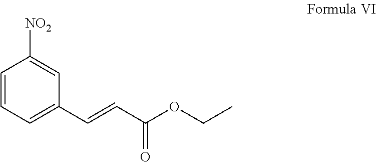 [SMILES: CCOC(=O)/C=C/c1cccc([N+](=O)[O-])c1]

b) reduction of (E)-ethyl 3-(3-nitrophenyl)acrylate of formula VI with tin chloride-dihydrate in presence of suitable solvent to give (E)-ethyl 3-(3-aminophenyl)acrylate of formula V;

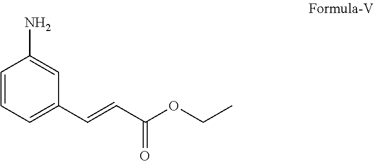

c) chlorosulfonation of (E)-ethyl 3-(3-aminophenyl)acrylate of formula V with Copper (I) chloride by passing sulphur dioxide gas in suitable acid and a suitable solvent to give (E)-ethyl 3-(3-(chlorosulfonyl)phenyl)acrylate of formula IV;

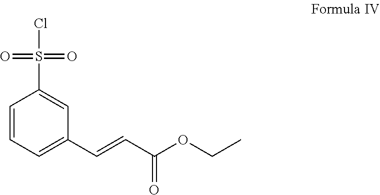 [SMILES: CCOC(=O)/C=C/c1cccc(S(=O)(=O)Cl)c1]

d) coupling of (E)-ethyl 3-(3-(chlorosulfonyl)phenyl)acrylate of formula IV with aniline in presence of suitable base to give (E)-3-(3-(N-phenylsulfamoyl)phenyl)acrylic acid of formula III;

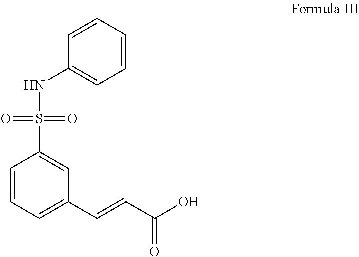 [SMILES: O=C(O)/C=C/c1cccc(S(=O)(=O)Nc2ccccc2)c1]

e) conversion of (E)-3-(3-(N-phenylsulfamoyl)phenyl)acrylic acid of formula III to acid chloride with suitable chlorinating agent in presence of dimethylformamide to give (E)-3-(3-(N-phenylsulfamoyl) phenyl) acryloyl chloride of formula II;

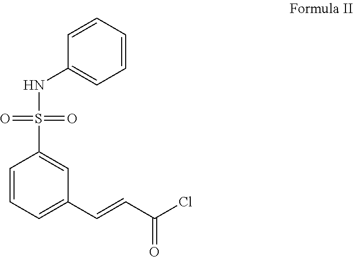 [SMILES: O=C(Cl)/C=C/c1cccc(S(=O)(=O)Nc2ccccc2)c1]

f) amino hydroxylation of (E)-3-(3-(N-phenylsulfamoyl) phenyl) acryloyl chloride of formula II with hydroxyl amine hydrochloride to give crude Belinostat of formula I; and

g) optionally, purifying the crude Belinostat of formula I by re-crystallizing from suitable solvent to provide pure Belinostat of formula I.

The suitable reagents/agents, solvents, acid and base are defined above and as below.

The suitable solvent used in the above reaction steps (a), (b), (c), (d), (e) and (f) may be selected from water, alcohols, hydrocarbons, aromatic hydrocarbons, chloro hydrocarbons, esters, ketones, ethers, nitriles, acetic acid or mixtures thereof.

The preferable solvent used in step a) is water, the preferable solvent used in step c) is aqueous acetic acid, preferable solvent used in step d) is dichloromethane, the preferable solvent used in step e) is dichloromethane and the preferable solvent used in step f) is tetrahydrofuran.

A preferred embodiment of the present invention provides a process for the preparation of Belinostat of formula I, comprising the steps of:
(a) reacting 3-nitrobenzaldehyde of formula VIII with suitable triethylphosphonoacetate in the presence of Potassium carbonate and water to provide (E)-ethyl 3-(3-nitrophenyl)acrylate of formula VI;
(b) reducing (E)-ethyl 3-(3-nitrophenyl)acrylate of formula VI with tin chloride-dihydrate in presence of ethanol solvent to give compound of formula V;
(c) chlorosulfonating (E)-ethyl 3-(3-aminophenyl)acrylate of formula V with copper (I) chloride by passing sulphur dioxide gas in presence of mixture of acetic acid and hydrochloric acid to provide formula IV;
(d) coupling (E)-ethyl 3-(3-(chlorosulfonyl)phenyl)acrylate of formula IV with aniline in the presence of 4-dimethylaminopyridine to provide formula III;
(e) chlorinating (E)-3-(3-(N-phenylsulfamoyl)phenyl)acrylic acid of formula III with oxalylchloride in the presence of dimethylformamide to give formula II;
(f) amino hydroxylating(E)-3-(3-(N-phenylsulfamoyl) phenyl) acryloyl chloride of formula II with hydroxyl amine hydrochloride to provide Belinostat of formula I.

The above process further optionally comprises a purification step (g).

In another aspect the present invention provides a process for the purification of Belinostat comprising the steps of:
(a) suspending crude Belinostat of formula I in suitable solvents like ethyl acetate, methanol, ethanol, isopropanol, n-butanol, water, methyl tertiary butyl ether, acetone, diethyl ether, isopropyl ether, dichloromethane, acetonitrile, acetic acid and tetrahydrofuran, toluene, cyclohexane solvents or mixtures thereof;
(b) heating the reaction mixture to a temperature ranging from 25°-65° C. for 1 to 5 hrs;
(c) cooling the reaction mixture to 0-5° C.;
(d) filtering the solid separated under vacuum; and
(e) optionally, washing the solid with the mixture of suitable solvents like, ethyl acetate, methanol, ethanol, isopropanol, n-butanol, water, methyl tertiary butyl ether, acetone, diethyl ether, isopropyl ether, dichloromethane, acetonitrile, acetic acid and tetrahydrofuran, toluene, cyclohexane solvents or mixtures thereof at 0-5° C.

In one preferred embodiment, the suitable solvent used in step (a) is a mixture of solvents selected from combinations: ethyl acetate and methanol (4:1) mixture, ethanol and ethyl acetate mixture (2:4) mixture, isopropyl alcohol and ethyl acetate (3:4) mixture, dichloromethane and ethyl acetate (0.5:6) mixture and Isopropyl alcohol and Tetrahydrofuran (1:3) mixture.

Belinostat obtained by the purification process of the present invention is having purity of greater than 98%, preferably greater than 99%.

Belinostat obtained by the process of the present invention is having impurities less than 0.05% (w/w) by HPLC, preferably less than 0.03% (w/w) by HPLC.

In another aspect, substantially pure Belinostat compound of formula I having purity of greater than 98%, preferably greater than 99% and impurities less than 0.05% (w/w), preferably less than 0.03% (w/w) by HPLC is provided.

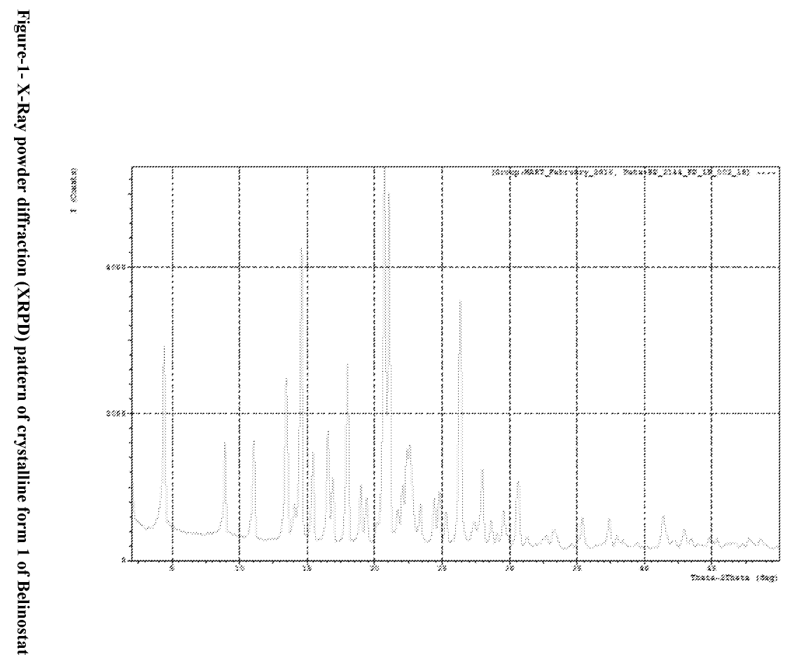

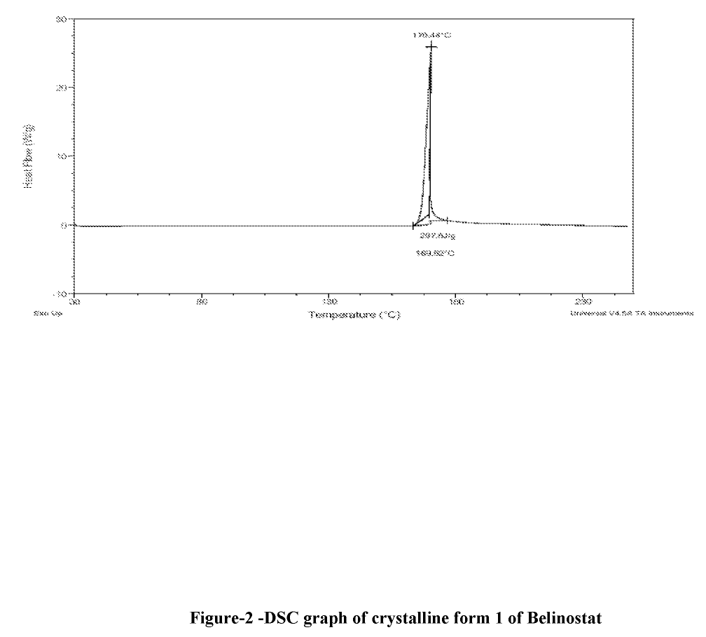

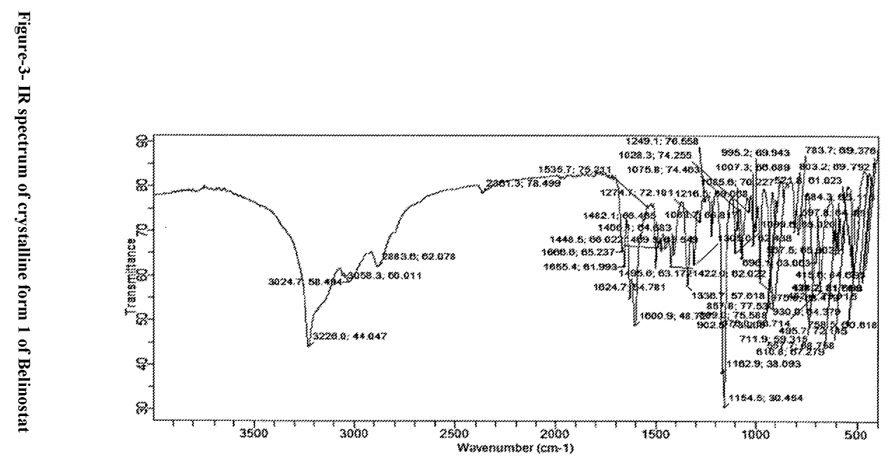

The pure Belinostat of formula I obtained after purification is in crystalline form designated as form 1, characterized by an X-ray diffraction pattern comprising one or more of the reflections at: 4.5, 13.5, 14.6, 18.0, 20.7, 21.1 and 26.3±0.2 degrees 2 theta; which is further characterized by an X-ray powder diffraction pattern comprising one or more of the reflections at: 8.9, 11.1, 15.4, 16.5, 22.4, 22.6 & 28.0±0.2 degrees 2 theta values as depicted in XRPD FIG. 1 and tabulated in Table-1 below; identified by the DSC thermogram as depicted in FIG. 2; and IR spectrum as depicted in FIG. 3.

In another aspect the invention provides a novel crystalline form 1 of Belinostat characterized by XRPD pattern of FIG. 1, DSC thermogram of FIG. 2 and IR spectrum of FIG. 3. The novel form 1 is obtained by the re-crystallization in solvent or solvent mixture as described above.

The crystalline form 1 of Belinostat may produce an X-ray diffraction pattern comprising one or more of the following reflections at: 4.5, 13.5, 14.6, 18.0, 20.7, 21.1 and 26.3±0.2 degrees 2 theta or that produces an X-ray powder diffraction pattern further comprising one or more of the following reflections at: 8.9, 11.1, 15.4, 16.5, 22.4, 22.6 & 28.0±0.2 degrees 2 theta or that produces an X-ray powder diffraction pattern comprising the 2 theta values tabulated in Table-1.

The crystalline form 1 of Belinostat may also be identified by the DSC thermogram as depicted in FIG. 2; and IR spectrum as depicted in FIG. 3.

TABLE 1
X-Ray diffraction data of polymorphic form 1 of Belinostat

| Peak no. | 2 Theta (deg) | d(A) | I/I1 |
|---|---|---|---|
| 1 | 4.57 | 19.28 | 48 |
| 2 | 8.72 | 10.12 | 3 |
| 3 | 8.99 | 9.82 | 24 |
| 4 | 11.11 | 7.95 | 26 |
| 5 | 13.52 | 6.54 | 42 |
| 6 | 14.11 | 6.26 | 9 |
| 7 | 14.62 | 6.05 | 78 |
| 8 | 15.47 | 5.72 | 23 |
| 9 | 16.59 | 5.33 | 30 |
| 10 | 16.94 | 5.22 | 17 |
| 11 | 17.73 | 4.99 | 3 |
| 12 | 18.04 | 4.91 | 48 |
| 13 | 19.03 | 4.65 | 15 |
| 14 | 19.45 | 4.55 | 12 |
| 15 | 20.21 | 4.38 | 4 |
| 16 | 20.77 | 4.27 | 100 |
| 17 | 21.10 | 4.20 | 94 |
| 18 | 21.75 | 4.08 | 7 |
| 19 | 22.12 | 4.01 | 14 |
| 20 | 22.41 | 3.96 | 23 |
| 21 | 22.63 | 3.92 | 24 |
| 22 | 22.92 | 3.87 | 9 |
| 23 | 23.42 | 3.79 | 10 |
| 24 | 24.45 | 3.63 | 12 |
| 25 | 24.82 | 3.58 | 14 |
| 26 | 25.32 | 3.51 | 8 |
| 27 | 26.37 | 3.37 | 66 |
| 28 | 27.39 | 3.25 | 5 |
| 29 | 27.71 | 3.21 | 5 |
| 30 | 28.0 | 3.18 | 20 |
| 31 | 28.67 | 3.11 | 6 |
| 32 | 29.11 | 3.06 | 3 |
| 33 | 29.58 | 3.01 | 9 |
| 34 | 30.64 | 2.91 | 17 |
| 35 | 33.35 | 2.68 | 5 |
| 36 | 35.40 | 2.53 | 9 |
| 37 | 37.40 | 2.40 | 9 |
| 38 | 37.95 | 2.36 | 3 |
| 39 | 41.40 | 2.17 | 9 |
| 40 | 42.95 | 2.10 | 5 |

The following examples further illustrate the present invention, but should not be construed in any way as to limit its scope.

### EXAMPLE-1: PROCESS FOR THE PREPARATION OF (E)-ETHYL 3-(3-NITROPHENYL) ACRYLATE (VI)

100 g of 3-nitrobenzaldehyde (VIII) was charged into round bottom flask containing 265.8 g potassium carbonate and 300 ml of DM water at 25-30° C. The temperature of the reaction mass was brought to 20-25° C. and then slowly 272 g of triethylphosphonoacetate (VII) added for a period of 20-30 min and maintained for 2-3 h. After completion of reaction, the solid was filtered under vacuum at 25-30° C. and washed with 300 ml of 1N hydrochloric acid followed by 300 ml of DM water. The obtained solid was dried at 50-55° C. for 8-10 h to get the title compound VI. Yield: 82-88%; Purity: 97.68%

### EXAMPLE-2: PROCESS FOR THE PREPARATION OF (E)-ETHYL 3-(3-AMINOPHENYL) ACRYLATE (V)

100 g of (E)-ethyl 3-(3-nitrophenyl) acrylate (VI) was dissolved in 1000 ml of ethanol and 346.7 g of tin (II) chloride dihydrate was added for 20-30 minutes at 25-30° C. The reaction mass was maintained for 3.5 hrs at 80-82° C. and cooled to 25-30° C. The pH of the reaction mass was neutralized with 150 g of sodium carbonate. The reaction mass was quenched with 1200 ml of water, filtered the salts and washed with dichloromethane. The filtrate was extracted with dichloromethane, aqueous and organic layers were separated; Washed the dichloromethane layerwith aqueous sodium chloride solution and dried over sodium sulphate. Organic layer was distilled under vacuum below 40° C. The obtained crude was dissolved in 180 ml of cyclohexane at 25-30° C. and stirred for 20-30 min at 8-10° C. The solid so obtained was washed with 20 mL of cyclohexane and dried below 40° C.

Yield: 75-80%; Purity by HPLC:98.25%

### EXAMPLE-3: PROCESS FOR THE PREPARATION OF (E)-ETHYL 3-(3-CHLOROSULFONYL) PHENYL ACRYLATE (IV)

To 100 g of (E)-ethyl 3-(3-aminophenyl) acrylate (V), 480 ml of acetic acid and 140 ml of HCl at 25-30° C. were added and cooled to −5 to 0° C.An aqueous sodium nitrite solution was added to reaction mass at −5 to 0° C. and maintained for 30-40 min. To this a mixture of 600 ml of acetic acid and 25 g of copper (I) chloride solution saturated with sulphur dioxide gas was added for 5 min at −5 to 0° C. The reaction mass was maintained for 30-45 min at 5 to 0° C. The reaction mass was quenched with 2.5 kg of ice and stirred for 20-30 min at −5 to 0° C. The resulting solid was filtered and dried under vacuum.

Yield: 60-65%; Purity by HPLC: 96.47%

### EXAMPLE-4: PROCESS FOR THE PREPARATION OF (E)-3-(3-(N-PHENYLSULFAMOYL)PHENYL)AACRYLIC ACID (III)

To a mixture of 450 ml of dichloromethane, 38.93 g of aniline and 60 g of 4-dimethylaminopyridine, 100 g of (E)-ethyl 3-(3-aminophenyl) acrylate (IV) dissolved in 350 ml dichloromethane over a period of 20-30 min at 25-30° C. was added. After completion of the reaction, the reaction mass was washed with 2 N hydrochloric acid and the organic and aqueous layers are separated. To the organic layer, aqueous sodium hydroxide solution was charged and stirred for 8 hrs at 25-30° C. The aqueous layer was washed with dichloromethane and treated with neutral charcoal. The pH of the aqueous layer was adjusted to 1 to 2 using concentrated hydrochloric acid and stirred for 20-30 minutes at 25-30° C. The resulting solid was filtered and dried under vacuum.

Yield: 63-72%; Purity by HPLC: 98.32%

### EXAMPLE-5: PROCESS FOR THE (E)-N-HYDROXY-3-(3-PHENYLSULFAMOYL-PHENYL)-ACRYLAMIDE (I)

100 g of (E)-3-(3-(N-phenylsulfamoyl) phenyl) acrylic acid (III) was dissolved in dichloromethane and 0.2 ml of dimethyl formamide. 90 ml of oxalyl chloride was slowly added to the reaction mass at 25-30° C. The reaction mass was stirred for 2-3 hrs and the solvent was distilled off under vacuum. To the obtained crude, tetrahydrofuran was added and kept aside. In another RB flask, hydroxyl amine hydrochloride is dissolved in tetrahydrofuran and aqueous sodium carbonate solution was taken, above acid chloride solution and sodium carbonate solution were added simultaneously at 25-30° C. The reaction mass was stirred for 30 minutes and ethyl acetate was added. The organic layer was separated and washed with water and distilled off the solvent under vacuum. The obtained crude was taken in ethyl acetate and stirred for 10-12 hrs at 25-30° C., filtered and dried the solid under vacuum.

### Purification of (E)-N-Hydroxy-3-(3-Phenylsulfamoyl-Phenyl)-Acrylamide (I) or Belinostat

The above crude solid was suspended in a mixture of ethyl acetate and methanol (4:1) and stirred the suspension for 15 hrs at 25-30° C. The reaction mixture was cooled to 0-5° C. and maintained for 4 hrs. The obtained solid was filtered under vacuum and washed with 0.5 volumes of chilled 4:1 mixture of ethyl acetate and methanol solution.

The resulting solid was dried under vacuum.

Yield: 80-85%, Purity by HPLC: 99.0%

The crude Belinostat was treated with a mixture of ethanol and ethyl acetate mixture (2:4) and the suspension was stirred for 1 hr at 50-60° C. The reaction mixture was cooled to 0-5° C. and maintained for 2 hr. The obtained solid was filtered under vacuum and washed with 0.5 volumes of chilled 2:4 mixture of ethanol and ethyl acetate solution. The resulting solid was dried under vacuum.

Yield: 75-80%, Purity by HPLC: 99.2%

The crude Belinostat was treated with a mixture of isopropyl alcoholand ethyl acetate mixture (3:4) and stirred the suspension for 1 hr at 60-65° C. The reaction mixture is cooled to 0-5° C. and maintained for 1 hr. The obtained solid was filtered under vacuum and washed with 0.5 volumes of chilled 3:4 mixture of IPA and ethyl acetate solution.

The resulting crystalline solid was dried under vacuum.

Yield: 78-82%; Purity by HPLC: 99.0%

The crude Belinostat was treated with a mixture of dichloromethane and ethyl acetate mixture (0.5:6) and stirred the suspension for 1 hr at 40-45° C. The reaction mixture is cooled to 0-5° C. and maintained for 1 hr. The obtained solid was filtered under vacuum and washed with 0.5 volumes of chilled acetate at 0-5° C. The resulting crystalline solid was dried under vacuum.

Yield: 56-60%; Purity by HPLC: 99.91%

To the crude Belinostat, 4 Volumes of 1:3 mixture of Isopropyl alcohol and Tetrahydrofuran was added at 25-30° C. and stirred for 25-30 minutes at 25-30° C. Then cooled the solution to 10-15° C. and maintained for 20-30 min at 10-15° C. Then filtered and washed the solid with 1 volume of 1:3 mixture of Isopropyl alcohol and tetrahydrofuran.

Yield: 48 to 50%; Purity: 99.91%

1. A process for the preparation of Belinostat of formula I:
comprising the steps of: 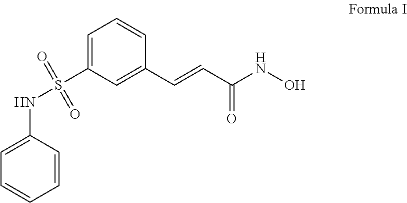 [SMILES: O=C(/C=C/c1cccc(S(=O)(=O)Nc2ccccc2)c1)NO]
(a) reacting 3-nitrobenzaldehyde of formula VIII with a Wittig reagent to provide (E)-ethyl 3-(3-nitrophenyl)acrylate of formula VI; 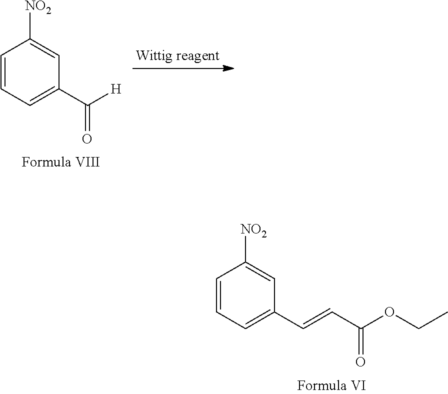 [SMILES: O=Cc1cccc([N+](=O)[O-])c1 ; CCOC(=O)/C=C/c1cccc([N+](=O)[O-])c1]
(b) reducing (E)-ethyl 3-(3-nitrophenyl)acrylate of formula VI with a reducing agent to provide (E)-ethyl 3-(3-aminophenyl)acrylate of formula V; 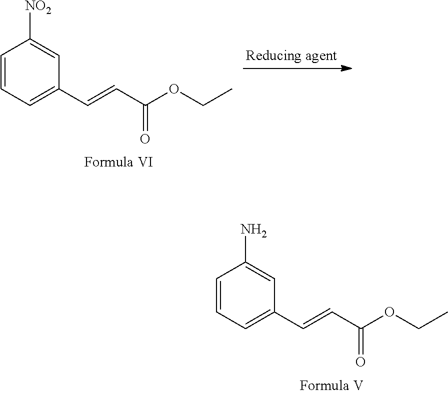 [SMILES: CCOC(=O)/C=C/c1cccc([N+](=O)[O-])c1 ; CCOC(=O)/C=C/c1cccc(N)c1]
(c) diazotizing (E)-ethyl 3-(3-aminophenyl)acrylate of formula V followed by chlorosulphonating with a chlorosulphonating reagent to provide (E)-ethyl 3-(3-(chlorosulfonyl)phenyl)acrylate of formula IV; 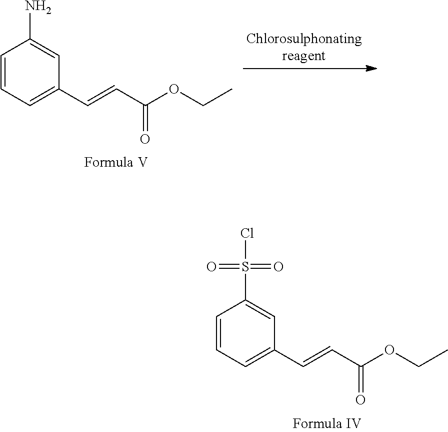 [SMILES: CCOC(=O)/C=C/c1cccc(N)c1 ; CCOC(=O)/C=C/c1cccc(S(=O)(=O)Cl)c1]
(d) coupling (E)-ethyl 3-(3-(chlorosulfonyl)phenyl)acrylate of formula IV with aniline to provide (E)-3-(3-(N-phenyl sulfamoyl)phenyl)acrylic acid of formula III; 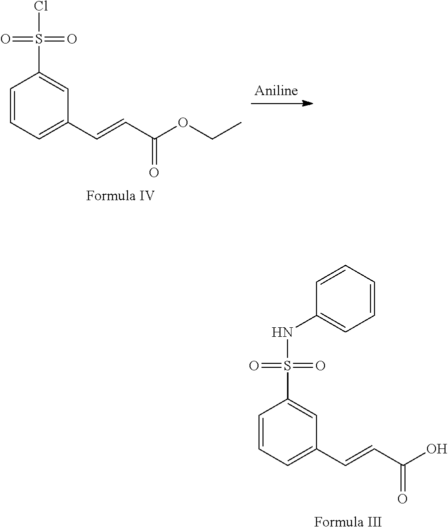 [SMILES: CCOC(=O)/C=C/c1cccc(S(=O)(=O)Cl)c1 ; O=C(O)/C=C/c1cccc(S(=O)(=O)Nc2ccccc2)c1]
(e) converting (E)-3-(3-(N-phenylsulfamoyl)phenyl)acrylic acid of formula III to acid chloride with a chlorinating agent to provide (E)-3-(3-(N-phenylsulfamoyl) phenyl) acryloyl chloride of formula II; 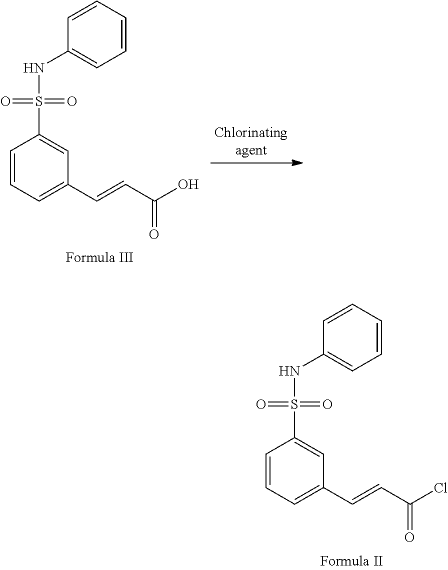 [SMILES: O=C(O)/C=C/c1cccc(S(=O)(=O)Nc2ccccc2)c1 ; O=C(Cl)/C=C/c1cccc(S(=O)(=O)Nc2ccccc2)c1]
(f) amino hydroxylating (E)-3-(3-(N-phenylsulfamoyl) phenyl) acryloyl chloride of formula II with hydroxyl amine hydrochloride to provide crude Belinostat of formula I; and 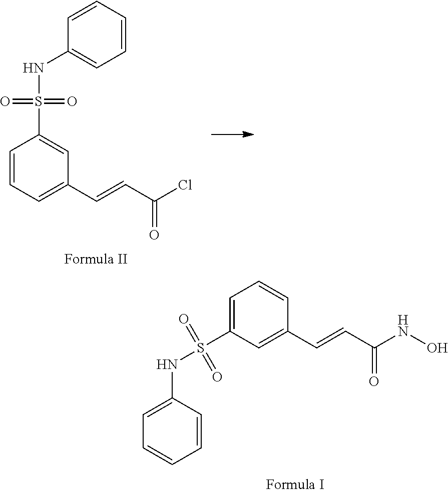 [SMILES: O=C(Cl)/C=C/c1cccc(S(=O)(=O)Nc2ccccc2)c1 ; O=C(/C=C/c1cccc(S(=O)(=O)Nc2ccccc2)c1)NO]
(g) optionally, purifying the compound of formula I by re-crystallizing from suitable solvents to provide substantially pure Belinostat of formula I.

2. The process as claimed in claim 1, wherein the Wittig reagent in step (a) is selected from the group comprising of Ethyl (triphenylphosphoranylidene) acetate, tert-Butoxycarbonylmethylene) triphenylphosphorane, benzyl (tri phenylphosphoranylidene) acetate, methyl (triphenylphosphoranylidene) acetate, Triethylphosphonoacetate and trimethylphosphonoacetate.

3. The process as claimed in claim 1, wherein step (a) is carried out in the presence of a base comprising of inorganic base selected from caesium carbonate (Cs₂CO₃), potassium carbonate (K₂CO₃), sodium carbonate (Na₂CO₃),silver carbonate (Ag₂CO₃),tripotassium phosphate (K₃PO₄), sodium hydroxide (NaOH), potassium hydroxide (KOH), caesium hydroxide (CsOH), potassium methoxide (KOMe), sodium methoxide (NaOMe),sodium ethoxide (NaOEt), lithium tert-butoxide (LiOtBu), sodium tert-butoxide (NaOtBu),potassium tert-butoxide (KOtBu), Sodium bis(trimethylsilyl)amide (NaHMDS), potassium bis(trimethylsilyl)amide (KHMDS), sodium hydride (NaH); or organic base selected from pyridine, triethyl amine, leutidine, (1,4-diazabicyclo[2.2.2]octane) (DABCO), 1,8-Diazabicyclo[5.4.0]undec-7-ene (DBU), N,N-diisopropylethylamine (DIPEA), butyl lithium (BuLi), lithium diisopropylamide (LDA), 1,2,2,6,6-pentamethylpipiridine, 1,1,3,3-tetramethylguanidine, diispropyl ethyl amine (iPr₂Net) and tributyl amine (NBu₃).

4. The process as claimed in claim 1, wherein the reducing agent in step (b) is Tin chloride-dihydrate.

5. The process as claimed in claim 1, wherein the chlorosulphonating reagent in step (c) is selected from copper (I) chloride or copper (II) chloride.

6. The process as claimed in claim 1, wherein the chlorosulphonating reagent in step (c) is used in combination with sulphur dioxide gas.

7. The process as claimed in claim 1, wherein step (c) is carried out in presence of hydrochloric acid (HCl).

8. The process as claimed in claim 1, wherein the reaction temperature in step (c) ranges from −10 to 0° C.

9. The process as claimed in claim 1, wherein step (d) is carried out in presence of a base selected from triethyl amine, pyridine, metal carbonates, metal hydroxides, metal alkoxides and dimethyl amino pyridine.

10. The process as claimed in claim 1, wherein the acid chlorinating agent in step (e) is selected from thionyl chloride and oxalylchloride.

11. The process as claimed in claim 1, wherein one or more of the process steps (a), (b), (c), (d), (e) and (f) can be carried out in presence of a solvent selected from water, alcohols, hydrocarbons, aromatic hydrocarbons, chloro hydrocarbons, esters, ketones, ethers, nitriles, acetic acid or mixtures thereof.

12. The process as claimed in claim 11, wherein the step (a) is carried out in presence of solvent water.

13. The process as claimed in claim 11, wherein the step (b) is carried out in presence of an alcohol solvent selected from ethanol, methanol, propanol, isopropanol and mixtures thereof.

14. The process as claimed in claim 11, wherein the step (c) is carried out in presence of solvent aqueous acetic acid (Aq. AcOH).

15. The process as claimed in claim 11, wherein the step (d) is carried out in presence of solvent dichloromethane (DCM).

16. The process as claimed in claim 11, wherein the step (e) is carried out in presence of solvent dichloromethane (DCM).

17. The process as claimed in claim 11, wherein the step (f) is carried out in presence of solvent tetrahydrofuran (THF).

18. The process as claimed in claim 1, wherein the solvent in step (g) is selected from alkanes, alcohols, water, acetonitrile, tetrahydrofuran, acetone, ethyl acetate, dichloromethane, methyl tertiary butyl ether, diethyl ether, isopropyl ether, acetic acid or mixtures thereof.

19. The process as claimed in claim 1, wherein the crude Belinostat of formula I is obtained by the process, comprising the steps of:
(g) reacting 3-nitrobenzaldehyde of formula VIII with triethylphosphonoacetate in the presence of Potassium carbonate and water to provide (E)-ethyl 3-(3-nitrophenyl)acrylate of formula VI;
(h) reducing (E)-ethyl 3-(3-nitrophenyl)acrylate of formula VI with tin chloride-dihydrate in presence of ethanol solvent to provide compound of formula V;
(i) chlorosulfonating (E)-ethyl 3-(3-aminophenyl)acrylate of formula V with copper (I) chloride by passing sulphur dioxide gas in presence of mixture of acetic acid and hydrochloric acid to provide formula IV;
(j) coupling (E)-ethyl 3-(3-(chlorosulfonyl)phenyl)acrylate of formula IV with aniline in the presence of 4-dimethylaminopyridine to provide formula III;
(k) chlorinating (E)-3-(3-(N-phenylsulfamoyl)phenyl)acrylic acid of formula III with oxalylchloride in the presence of dimethylformamide to give formula
(l) amino hydroxylating (E)-3-(3-(N-phenylsulfamoyl) phenyl) acryloyl chloride of formula II with hydroxyl amine hydrochloride to provide crude Belinostat of formula I.

20. The process as claimed in claim 1, wherein the purification by re-crystallization in step (g) comprises following process steps:
(f) suspending crude Belinostat of formula I in suitable solvents selected from ethyl acetate, methanol, ethanol, isopropanol, n-butanol, water, methyl tertiary butyl ether, acetone, diethyl ether, isopropyl ether, dichloromethane, acetonitrile, acetic acid and tetrahydrofuran, toluene, cyclohexane solvents or mixtures thereof;
(g) heating the reaction mixture to a temperature between 25°-65° C. for 1 to 5 hrs;
(h) cooling the reaction mixture to 0-5° C.;
(i) filtering the solid separated under vacuum; and
(j) optionally, washing the solid with the mixture of suitable solvents selected from ethyl acetate, methanol, ethanol, isopropanol, n-butanol, water, methyl tertiary butyl ether, acetone, diethyl ether, isopropyl ether, dichloromethane, acetonitrile, acetic acid and tetrahydrofuran, toluene, cyclohexane solvents or mixtures thereof at 0-5° C.

21. The process as claimed in claim 1, wherein the Belinostat obtained after purification process is substantially pure having purity of greater than 98% and impurities less than 0.05% (w/w) by HPLC.

22. The process as claimed in claim 1, wherein the Belinostat obtained after purification process is substantially pure having purity of greater than 99% and impurities less than 0.03% (w/w) by HPLC.

23. The process as claimed in claim 1 or claim 20, wherein the pure Belinostat of formula I obtained after purification is in crystalline form designated as form 1, characterized by an X-ray diffraction pattern comprising one or more of the reflections at: 4.5, 13.5, 14.6, 18.0, 20.7, 21.1 and 26.3±0.2 degrees 2 theta; which is further characterized by an X-ray powder diffraction pattern comprising one or more of the reflections at: 8.9, 11.1, 15.4, 16.5, 22.4, 22.6 & 28.0±0.2 degrees 2 theta.

24. A process for the purification of Belinostat of formula I, comprising the steps of:
(f) suspending crude Belinostat of formula I in suitable solvents selected from ethyl acetate, methanol, ethanol, isopropanol, n-butanol, water, methyl tertiary butyl ether, acetone, diethyl ether, isopropyl ether, dichloromethane, acetonitrile, acetic acid and tetrahydrofuran, toluene, cyclohexane solvents or mixtures thereof;
(g) heating the reaction mixture to a temperature ranging from 25°-65° C. for 1 to 5 hrs;
(h) cooling the reaction mixture to 0-5° C.;
(i) filtering the solid separated under vacuum; and
(j) optionally, washing the solid with the mixture of suitable solvents selected from ethyl acetate, methanol, ethanol, isopropanol, n-butanol, water, methyl tertiary butyl ether, acetone, diethyl ether, isopropyl ether, dichloromethane, acetonitrile, acetic acid and tetrahydrofuran, toluene, cyclohexane solvents or mixtures thereof at 0-5° C.

25. The process for purification as claimed in claim 24, wherein the suitable solvent used in step (a) is a mixture of solvents selected from combinations: ethyl acetate and methanol (4:1) mixture, ethanol and ethyl acetate mixture (2:4) mixture, isopropyl alcohol and ethyl acetate (3:4) mixture, dichloromethane and ethyl acetate (0.5:6) mixture and Isopropyl alcohol and Tetrahydrofuran (1:3) mixture.

26. The process for purification as claimed in claim 24, wherein the pure Belinostat of formula I obtained after purification is in crystalline form designated as form 1, characterized by an X-ray diffraction pattern comprising one or more of the reflections at: 4.5, 13.5, 14.6, 18.0, 20.7, 21.1 and 26.3±0.2 degrees 2 theta; which is further characterized by an X-ray powder diffraction pattern comprising one or more of the reflections at: 8.9, 11.1, 15.4, 16.5, 22.4, 22.6 & 28.0±0.2 degrees 2 theta.

27. Crystalline Belinostat of formula I designated as crystalline form 1, characterized by an X-ray diffraction pattern comprising one or more of the reflections at: 4.5, 13.5, 14.6, 18.0, 20.7, 21.1 and 26.3±0.2 degrees 2 theta; which is further characterized by an X-ray powder diffraction pattern comprising one or more of the reflections at: 8.9, 11.1, 15.4, 16.5, 22.4, 22.6 & 28.0±0.2 degrees 2 theta.

28. Substantially pure Belinostat compound of formula I having purity of greater than 98% and impurities less than 0.05% (w/w) by HPLC.

29. The process as claimed in claim 20, wherein the pure Belinostat of formula I obtained after purification is in crystalline form designated as form 1, characterized by an X-ray diffraction pattern comprising one or more of the reflections at: 4.5, 13.5, 14.6, 18.0, 20.7, 21.1 and 26.3±0.2 degrees 2 theta; which is further characterized by an X-ray powder diffraction pattern comprising one or more of the reflections at: 8.9, 11.1, 15.4, 16.5, 22.4, 22.6 & 28.0±0.2 degrees 2 theta.
